(19) United States
(12) Patent Application Publication
(10) Pub. No.: US 2019/0374172 A1
(43) Pub. Date: Dec. 12, 2019

(54) BIOLOGICAL INFORMATION RECORDING APPARATUS, SYSTEM, METHOD AND PROGRAM

(71) Applicants: OMRON CORPORATION, Kyoto (JP); OMRON HEALTHCARE CO., LTD., Kyoto (JP)

(72) Inventors: Natsuko HORIGUCHI, Kyoto (JP); Hiroshi NAKAJIMA, Kyoto-shi (JP); Tomohiro KUKITA, Kyoto (JP); Hirotaka WADA, Kyoto-shi (JP); Tamio UEDA, Takatsuki-shi (JP)

(21) Appl. No.: 16/545,167

(22) Filed: Aug. 20, 2019

Related U.S. Application Data
(63) Continuation of application No. PCT/JP2018/009565, filed on Mar. 12, 2018.

(30) Foreign Application Priority Data
Mar. 15, 2017 (JP) 2017-050603

(51) Int. Cl. A61B 5/00; A61B 5/0205

(52) U.S. Cl. CPC A61B 5/7285; A61B 5/0205; A61B 5/021; A61B 5/0002; A61B 5/4818

(57) ABSTRACT

According to one embodiment, a biological information recording apparatus include a first acquisition unit, a second acquisition unit, a determination unit, and a control unit. The determination unit determines whether or not the biological information has varied outside of a preset normal range. The control unit records the biological information, if the biological information has varied outside of the normal range and if the motion information of the living body is not detected in a period corresponding to a variation period of the biological information. The biological information is a blood pressure of the living body detected for each pulse along with a heartbeat.

### CROSS-REFERENCE TO RELATED APPLICATIONS

This application is a Continuation Application of PCT Application No. PCT/JP2018/009565, filed Mar. 12, 2018 and based upon and claiming the benefit of priority from Japanese Patent Application No. 2017-050603, filed Mar. 15, 2017, the entire contents of all of which are incorporated herein by reference.

### FIELD

The present invention relates to a biological information recording apparatus, system, method and program for use in record of continuously measured biological information.

### BACKGROUND

With the advances in sensor technology that have allowed high-performance sensors to become readily available, application of biological information to the treatment for early detection of abnormalities in the body has gained increasing medical importance.

Conventionally, for example, there is a known device that stores data measured by a plurality of vital sensors in time synchronization on a server side (see, for example, Jpn. Pat. Appin. KOKAI Publication No. 2006-254948).

In recent years, a type of blood pressure monitor (for example, according to the tonometry method) has been realized that is capable of continuously measuring the user's blood pressure beat by beat, for example, simply by being wrapped around the user's wrist. Using such a blood pressure monitor makes it possible to continuously measure the blood pressure without imposing a heavy load on the user, and to detect an abnormality of the living body, such as blood pressure variation, at an early stage.

However, in order to continuously measure biological information, not only a blood pressure, a large-capacity storage device is required to record measurement data, leading to increased costs for the device and the like.

### SUMMARY

According to a first aspect of the present invention, a biological information recording apparatus includes: a biological information acquisition unit that acquires biological information detected by a biological sensor; a motion information acquisition unit that acquires motion information of a living body detected by a motion sensor; a variation determination unit that determines whether or not the biological information has varied outside of a preset normal range; and a recordation control unit that records the biological information if the biological information has varied outside of the normal range, and if the motion information of the living body is not detected in a period corresponding to a variation period of the biological information, and does not record the biological information if the biological information has varied outside of the normal range and if the motion information of the living body is detected in the period corresponding to the variation period of the biological information.

According to a second aspect of the present invention, the recordation control unit includes: a recordation start unit that starts recording of the biological information if the biological information varies outside of the normal range; and a deletion unit that deletes the recorded biological information if the motion information of the living body is detected in the period corresponding to the variation period of the biological information.

According to a third aspect of the present invention, a blood pressure of the living body detected continuously is used as the biological information.

According to a fourth aspect of the present invention, a waveform that changes in synchronization with a cardiac or respiratory cycle of the living body is continuously detected and used as the biological information.

According to a fifth aspect of the present invention, a motion of the living body detected by an acceleration sensor is used as the motion information of the living body.

According to a sixth aspect of the present invention, the biological information recording apparatus further includes a respiration information acquisition unit that acquires information representing a respiration state of the living body from a respiration sensor. The recordation control unit determines whether or not the variation of the biological information outside of the normal range is caused by a respiratory variation based on information representing a respiration state acquired from the respiration sensor; and performs a control of not recording the biological information if it is determined that the variation of the biological information outside of the normal range is caused by the respiratory variation.

According to a seventh aspect of the present invention, the biological information recording apparatus further includes a respiration information acquisition unit that acquires information representing a respiration state of the living body from a respiration sensor, the recordation control unit including an apnea period detection unit that detects an apnea period of the living body based on the information representing the respiration state acquired from the respiration sensor, and a recordation unit that records the blood pressure detected by the blood pressure sensor even if the motion sensor detects that the living body is not moving, and if the blood pressure detected by the blood pressure sensor rises in a respiration period immediately after an end of the apnea period detected by the apnea period detection unit.

According to the first aspect of the present invention, the biological information is recorded, if the biological information detected by the biological sensor has varied outside of the normal range and if the motion information of the living body is not detected in a period corresponding to a variation period of the biological information. On the other hand, the biological information is not recorded, even if the biological information has varied outside of the normal range and if the motion information of the living body is detected in the period corresponding to the variation period of the biological information. That is, variations in biological information not caused by the motion of the living body are reliably recorded, whereas variations in biological information caused by the motion of the living body are not recorded. Therefore, it is possible to record the biological information necessary to determine the condition of the living body without omission and not to record unnecessary biological information, so that the memory capacity can be reduced.

According to the second aspect of the present invention, when biological information has varied outside of the normal range, recording of the biological information is started, and thereafter, if the motion information of the living body is detected in the period corresponding to the variation period of the biological information, the recorded biological information is deleted. Therefore, even if the motion information of the living body cannot be acquired in real time, and even if processing of the acquired motion information of the living body cannot be performed in real time, substantially unnecessary biological information caused by the motion of the living body can be deleted.

According to the third aspect of the present invention, a blood pressure of the living body measured continuously is used as the biological information. For this reason, it is possible to reliably record, without loss, the abnormal variation of the blood pressure which is not caused by the motion of the living body. It should be noted that if the motion information of the living body measured continuously is used, information derived from the motion of the living body in biological information can be excluded from the information to be recorded without omission.

According to the fourth aspect of the present invention, a waveform that changes in synchronization with a cardiac or respiratory cycle of the living body is continuously detected and used as the biological information. In this way, it is possible to record only a portion necessary for diagnosing an abnormality of the living body, even for a waveform that changes in association with the cardiac or respiratory cycle, such as the electrocardiogram or pulse wave of the living body. Thus, the storage capacity of the storage device can be suppressed to a small capacity.

According to the fifth aspect of the present invention, the information detected by the acceleration sensor is used as the motion information of the living body. For this reason, it is possible to detect the motion or vibration of the living body in a linear direction.

According to the sixth aspect of the present invention, whether or not the variation in biological information is a respiratory variation is determined based on the respiration state of the living body. If it is determined that the variation is a respiratory variation, control is performed so as not to record the biological information. Therefore, even if no change in body position is detected, if the variation in biological information is a respiratory variation, the biological information cannot be stored, and this can further reduce the storage amount of the recordation device.

According to the seventh aspect of the present invention, an apnea period of the living body is detected based on the information representing the respiration state acquired from the respiration sensor. Then, even if the motion sensor detects that the living body is moving, if the increase in blood pressure detected in the respiration period immediately after an end of the apnea period is detected, the blood pressure is recorded. Therefore, so-called blood pressure surges, in which the blood pressure rises rapidly in association with sleep apnea, can be recorded without omission.

According to the aspects of the present invention, it is possible to provide a biological information recording apparatus, system, method, and program capable of recording a variation of biological information caused by an abnormality of a living body without omission, and not recording unnecessary biological information, thereby reliably determining an abnormality of the living body and reducing the amount of recorded data of the biological information.

### BRIEF DESCRIPTION OF DRAWINGS

FIG. 1 is a block diagram showing a biological information recording apparatus according to an embodiment.

FIG. 2 is a view showing a watch-type wearable terminal which is an example of the biological information recording apparatus of FIG. 1.

FIG. 3 is a view showing that the biological information recording apparatus of FIG. 1 connects to a smart device and the smart device connects to a server.

FIG. 4 is a flowchart describing an example of the operation of the biological information recording apparatus of FIG. 1.

FIG. 5A is a graph showing temporal variations in blood pressure value in the case of blood pressure surge due to apnea.

FIG. 5B is a graph showing three-axis accelerations and their combined acceleration at the same time as that in FIG. 5A.

FIG. 6A is a graph showing temporal variations in blood pressure value in the case of respiratory variations.

FIG. 6B is a graph showing three-axis accelerations and their combined acceleration at the same time as that in FIG. 6A.

FIG. 7A is a graph showing temporal variations in blood pressure value in the case of variations due to a change in body position.

FIG. 7B is a graph showing three-axis accelerations and their combined acceleration at the same time as that in

FIG. 7A.

### DETAILED DESCRIPTION

Hereinafter, a biological information recording apparatus, system, method and program of an embodiment according to the present invention will be described with reference to the drawings. In the embodiment described below, components assigned with the same reference numbers are assumed to perform similar operations, and redundant descriptions thereof will be omitted.

The present embodiments were made in view of the above circumstances, and an object of the present embodiments is to provide a biological information recording apparatus, system, method, and program capable of recording a variation of biological information caused by an abnormality of a living body without omission; and not recording unnecessary biological information not caused by an abnormality of the living body, thereby reliably determining a biological abnormality and reducing the amount of recorded data of biological information.

### First Embodiment

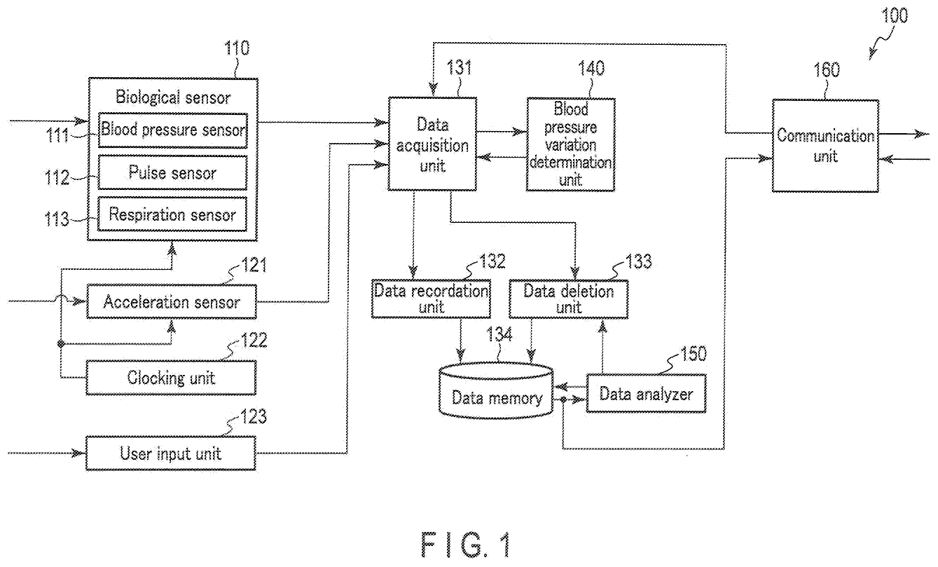

A biological information recording apparatus 100 according to the present embodiment will be described with reference to FIGS. 1, 2 and 3.

The biological information recording apparatus 100 includes a biological sensor 110, an acceleration sensor 121, a clocking unit 122, a user input unit 123, a data acquisition unit 131, a data recording unit 132, a data deletion unit 133, a data memory 134, a biological information variation determination unit 140, a data analyzer 150, and a communication unit 160. The biological sensor 110 includes a blood pressure sensor 111, a pulse sensor 112, and a respiration sensor 113.

The biological sensor 110 detects biological information from a living body, obtains time information from the clocking unit 122, and outputs biological information associated with the time. Examples of the biological information are a blood pressure, pulse, and respiration state. The blood pressure sensor 111 acquires a blood pressure value from the living body, and outputs the blood pressure value associated with the time that is continuously acquired from the clocking unit 122. The pulse sensor 112 acquires a pulse from the living body, and outputs a pulse rate associated with the time that is continuously acquired from the clocking unit 122. The respiration sensor 113 acquires information representing the state of expiration of the living body, that is, whether the living body is expiring and/or not expiring, and outputs information (also called respiration data) representing an expiration period associated with the time that is continuously acquired from the clocking unit 122.

In the present embodiment, the blood pressure sensor 111, the pulse sensor 112, and the respiration sensor 113 continuously detect biological information, for example, continue detection for 24 hours, and pass detection data to the data acquisition unit 131 in the next stage. However, the blood pressure sensor 111, the pulse sensor 112, and the respiration sensor 113 may measure periodically, not temporally continuously (for example, an oscillometric blood pressure measurement using a cuff is known). Alternatively, the blood pressure sensor 111 may perform a trigger measurement, in which measurement is triggered upon satisfaction of a predetermined condition. The trigger measurement does not measure temporally continuously; however, if the trigger is set appropriately, effective measurement can be performed, and an effect almost the same as that in the continuous measurement can be expected.

The acceleration sensor 121 is connected to the living body (for example, attached to the living body) to detect a motion of the living body. The acceleration sensor 121 of this embodiment detects the three-axis accelerations of the living body and passes the data to the data acquisition unit 131 of the next stage. The acceleration sensor 121 is not limited to the three-axis acceleration sensor. Besides the acceleration sensor that detects a linear motion, for example, an angular velocity sensor that detects a rotational motion, is known as a “motion sensor”. Therefore, it is possible to use a sensor capable of detecting accelerations of X, Y and Z axes and angular velocities of a roll axis, a pitch axis, and a yaw axis by combining the aforementioned sensors as appropriate. Alternatively, a three-axis angular velocity sensor capable of detecting angular velocities of a roll axis, a pitch axis, and a yaw axis may be used. The clocking unit 122 is configured to output the current time; for example, that is a normal clock. The clocking unit 122 may be set, for example, to obtain time calibration information from outside and output a correct time.

The user input unit 123 receives an instruction from the user and passes an instruction signal for operating the biological information recording apparatus 100 to the data acquisition unit 131. For example, the user input unit 123 receives an instruction for power on or off from the user, and turns on or off the biological information recording apparatus 100. The start of the measurement is triggered, for example, by the data acquisition unit 131 detecting that the pulse sensor 112 has started acquiring the pulse.

The data acquisition unit 131 acquires data from the biological sensor 110, the acceleration sensor 121, and the user input unit 123, passes a set of data to the biological information variation determination unit 140, and based on the determination result of the biological information variation determination unit 140, passes an instruction to the data recording unit 132 and/or the data deletion unit 133.

Based on the data from the data acquisition unit 131, the biological information variation determination unit 140 determines whether or not the blood pressure value has varied. For example, the biological information variation determination unit 140 determines that the blood pressure value has varied when the blood pressure value varies and deviates from a preset normal range. Alternatively, the biological information variation determination unit 140 may detect the amount of change of the blood pressure value per fixed time (differential value), and may determine that the blood pressure value has varied when the differential value exceeds a preset value. The biological information variation determination unit 140 determines not only a blood pressure variation, but also variations in pulse, electrocardiogram and respiration as needed.

The data recording unit 132 receives from the biological information variation determination unit 140, for example, via the data acquisition unit 131, notice of whether or not the blood pressure value of the living body has varied, and if it is determined that the blood pressure value of the living body has varied, the data acquisition unit 131 starts recording, in the data memory 134, data (biological information, such as a blood pressure value) acquired from the sensor 110 and data (acceleration data, such as three-axis accelerations) acquired from the acceleration sensor 121.

The data recording unit 132 records the biological information acquired by the data acquisition unit 131 from the biological sensor 110 in the data memory 134 together with time, within a period determined by the biological information variation determination unit 140 that the blood pressure value has varied. In addition to the above-mentioned biological information from biological sensor 110, the data recording unit 132 may also record biological information, such as the number of expirations and inspirations from the respiration sensor 113, in the data memory 134 together with time.

The data recording unit 132 records biological information in the data memory 134, for example, until the biological information variation determination unit 140 determines that the blood pressure value has returned to the normal range, and ceases recording of the biological information until the biological information variation determination unit 140 determines again that the blood pressure value has varied. Also in the case where recording of the biological information is started based on determination of the differential value of the blood pressure value, the recording of biological information is ended when it is determined that the blood pressure value of the biological information has returned to the normal range.

The data memory 134 stores at least the biological information received from the biological sensor 110 and the acceleration data from the acceleration sensor 121, together with time, in accordance with an instruction from the data recording unit 132. When the data deletion unit 133 instructs deletion of designated data, the data memory 134 deletes the designated data. The data memory 134 may further record at least one of respiration data and pulse data.

The data deletion unit 133 may delete the designated data from the data memory 134, for example, when instructed to delete the designated data from the user input unit 123.

The data analyzer 150 obtains time history of the biological information and the acceleration data stored in the data memory 134, and analyzes the biological information and acceleration data during a period in which blood pressure varies. The data analyzer 150 analyzes how the biological information and the acceleration data change with time; for example, it determines whether or not the change is a sudden change in blood pressure (blood pressure surge) due to apnea, a normal variation in blood pressure with respiration, or a variation due to a change of a body position. In addition, if respiration data is measured as biological information, the data analyzer 150 can more reliably identify whether or not apnea occurs than in the case of using acceleration data alone.

In the present embodiment, in the cases of a blood pressure variation not particularly abnormal due to respiration and a blood pressure variation due to a change in body position, the data analyzer 150 determines that the data does not represent a characteristic of the biological abnormality (the data is normal data). In this case, for example, the data analyzer 150 instructs the data deletion unit 133 to delete the biological information on the time of this blood pressure.

On the other hand, in the case where the data analyzer 150 identifies that a blood pressure surge due to apnea is a cause of the blood pressure variation, it determines the biological information to be distinctive data (abnormal data) representing biological abnormality, and does not delete but stores the biological information in the memory 134. This stored biological information is later subjected to an analysis to identify the cause of the blood pressure variation.

The communication unit 160 transmits, for example, data stored in the data memory 134 to an external server, or receives an instruction from an external device to activate or stop the biological information recording apparatus 100.

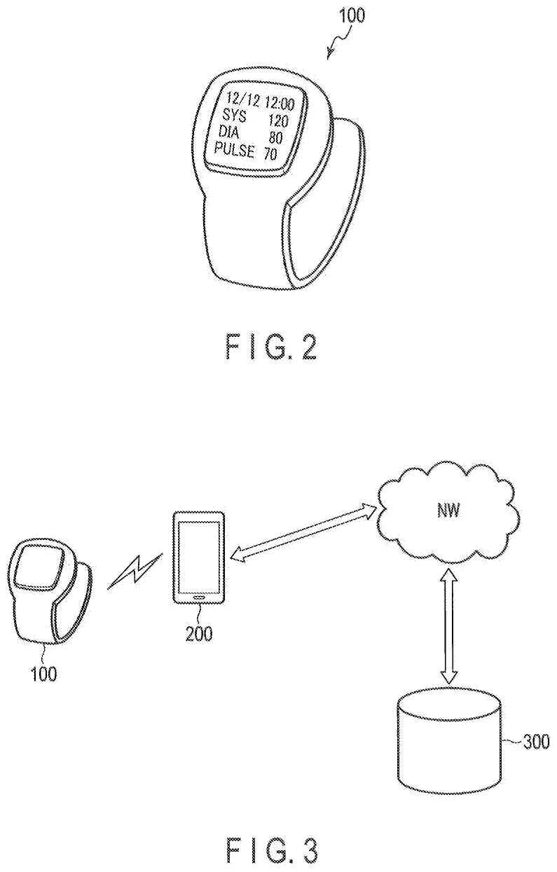

Next, an example of a specific device of the biological information recording apparatus 100 and the linkage with other devices will be described with reference to FIGS. 2 and 3.

The biological information recording apparatus 100 may have any form, but may be, for example, a watch-type wearable terminal shown in FIG. 2. This biological information recording apparatus 100 displays biological information, such as a systolic blood pressure, a diastolic blood pressure, and a pulse rate of the living body of the user, in addition to information displayed on a general clock, for example, today's date and current time. The biological information recording apparatus 100 can continuously measure the biological information of the user, for example, for each pulse along with a heartbeat, and can display the latest systolic blood pressure and diastolic blood pressure.

The biological information recording apparatus 100 may be connected to a smart device (typically, a smartphone, a tablet) 200 as illustrated in FIG. 3. The smart device 200 graphs and displays data transmitted by the biological information recording apparatus 100, and transmits the data to a server 300 via a network NW. The smart device 200 may have an application installed to manage data.

The server 300 accumulates data transmitted from the biological information recording apparatus 100 or the smart device 200. The server 300 may transmit data of the biological information of the user in response to access from a PC (Personal Computer) or the like disposed in a medical institution, for example, for use in a health guidance or diagnosis of the user.

Also, as described later, the server 300 may be a server 700 of a second embodiment. In this case, the server 300 may include the data analyzer 150 and the biological information variation determination unit 140. The server 300 transmits data to the biological information recording apparatus 100 or the smart device 200 to allow the user to browse.

Alternatively, the smart device 200 may include the data analyzer 150 and the biological information variation determination unit 140. In this case, the smart device 200 transmits data to be displayed on the biological information recording apparatus 100 to allow the user to browse. In addition, data may be browsed via the smart device 200.

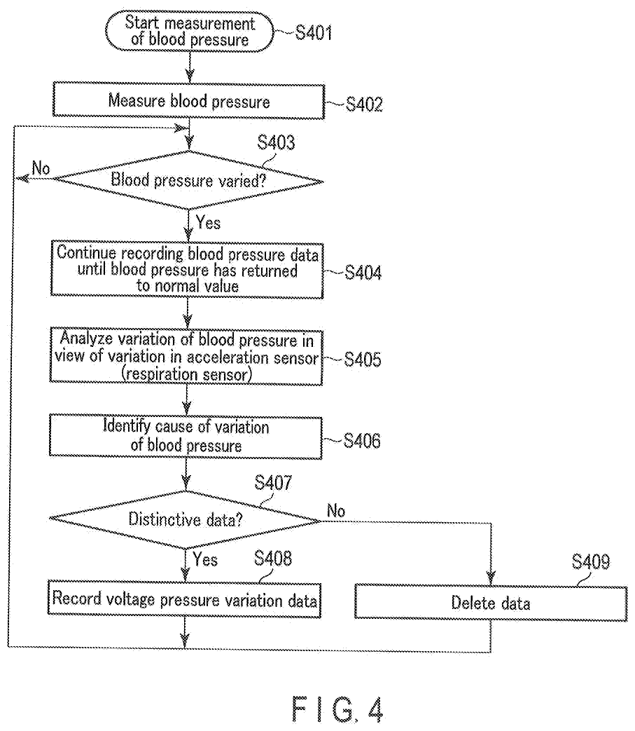

Next, the operation of the biological information recording apparatus 100 will be described with reference to FIG. 4.

(Step S401) The biological information recording apparatus 100 starts measurement of the blood pressure of a target living body. Specifically, the blood pressure sensor 111 starts the measurement of the blood pressure of the living body. The start of the measurement is triggered, for example, by the data acquisition unit 131 detecting that the pulse sensor 112 starts acquiring a pulse. Alternatively, the measurement of the blood pressure may be started in response to the user turning on the biological information recording apparatus 100 with the user input unit 123.

(Step S402) The blood pressure sensor 111 of the biological sensor 110 continues to measure the blood pressure. In the biological information recording apparatus 100 of the present embodiment, the blood pressure sensor 111 is a sensor capable of continuous measurement. For example, the blood pressure sensor 111 can continuously measure the blood pressure of the user for each pulse along with a heartbeat and continuously measure for 24 hours while the user is simply wearing it on the wrist. In this step, the blood pressure sensor 111 measures a blood pressure and passes data to the data acquisition unit 131. Although the biological information variation determination unit 140 also receives this data, it has not detected any variation in blood pressure value at this time point. Therefore, the value is not recorded in the data memory 134.

In other words, blood pressure time history data, respiration time history data, and living body acceleration data are recorded in the data memory 134 only after the blood pressure value starts varying. Therefore, among the blood pressure time history data, the respiration time history data, and the biological acceleration data, only data obtained when the biological information varies is recorded. Accordingly, the storage capacity of data memory 134 need not be unnecessarily constrained, and data resources can be used efficiently.

(Step S403) The biological information recording apparatus 100 determines whether or not the blood pressure value of the living body to be measured has varied. If it is determined that the blood pressure value has varied, the flow proceeds to step S404, and if it is determined that the blood pressure has not varied, the flow returns to step S402, and the blood pressure measurement is continued. For example, if the blood pressure value exceeds a preset normal range, the biological information recording apparatus 100 determines that the blood pressure value has changed. In the case of monitoring the variation amount per fixed time (differential value) of the blood pressure value, when the blood pressure value varies 10 mmHg or more per second, for example, it is determined that the blood pressure value has varied.

(Step S404) When it is determined that the blood pressure value has varied in step S403, the data recording unit 132 starts, from that point, a process of recording the biological information acquired by the data acquisition unit 131 in the data memory 134. Then, the process of recording the biological information is continued until the biological information determination unit 140 determines that the blood pressure value has returned to the normal range.

(Step S405) The data analyzer 150 analyzes the variation value of the blood pressure data stored in the data memory 134 in view of the acceleration data and the respiration data. The data analyzer 150 searches for a cause of a variation in blood pressure data.

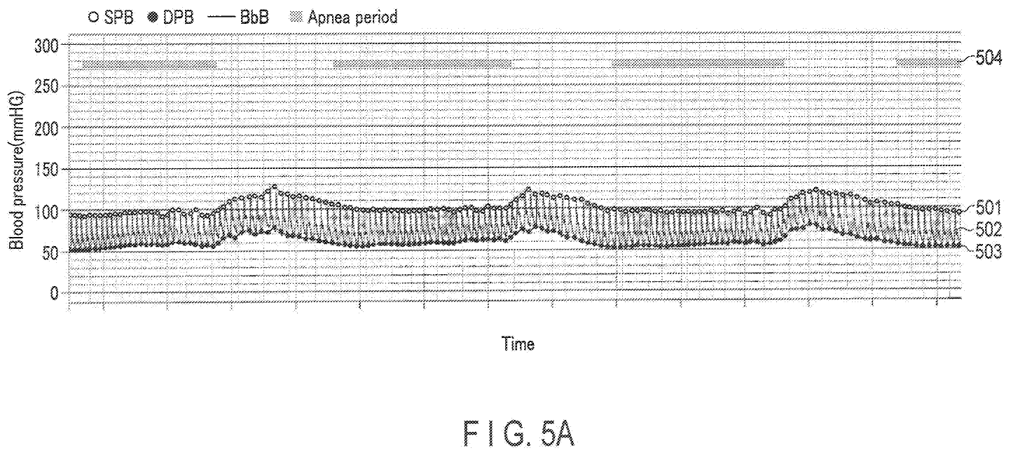

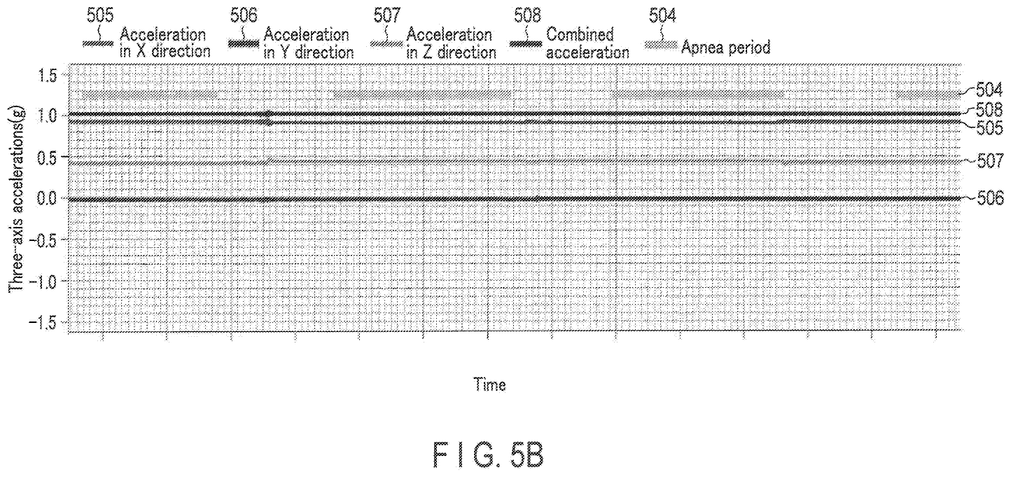

(Step S406) The data analyzer 150 compares the variation value of the blood pressure data with, for example, the acceleration data and the respiration data to identify the cause of the variation of the blood pressure data. In this example, whether or not the body position is moving is determined based on the variation of the acceleration data, whether or not there is no period of breathing stop (apnea) is determined based on the respiration data, etc. to identify the cause. Later, explanations will be given with reference to FIG. 5A and the subsequent figures.

(Step S407) The data analyzer 150 determines whether or not the blood pressure data that varies is distinctive data based on the cause of the variation of the blood pressure identified in Step S406. If it is determined that the varying blood pressure data is distinctive, the flow proceeds to step S408. If it is not determined that the data is distinctive, the flow proceeds to step S409. Here, blood pressure data that is distinctive data indicates that the time variation of the blood pressure value of the blood pressure data is not normal. A typical example is the case where the temporal variation of blood pressure values is caused by a disease, for example, obstructive sleep apnea syndrome (OSAS).

(Step S408) In the present embodiment, attention is paid to the case where the variation of blood pressure data is not normal. Therefore, in the case of blood pressure data caused by a disease, it is regarded as useful data that is worthy of attention. Thus, for example, the data analyzer 150 instructs the data recording unit 132 to record the blood pressure data and related data (for example, acceleration data and respiration data) in the data memory 134. Although the data have already been recorded in step S404, for example, the attribute is changed so as to permanently maintain the record in this step. Alternatively, the data may be stored in a temporary storage device in step S404 (for example, stored in a storage device having a high access speed but small capacity), and in step S408, the blood pressure data and associated data may be stored in, for example, a large-capacity storage device having a low access speed but higher reliability (the data memory 134 may include these two types of storage devices). Further, step S408 may be deleted; that is, the data memory 134 may be provided to perform step S404, in which case a plurality of storage devices are not provided.

(Step S409) In the present embodiment, attention is paid to the case where the variation of blood pressure data is not normal. Therefore, if it is determined that the change in blood pressure is simply due to, for example, a change in body position, the data deletion unit 133 deletes this blood pressure data and related data. In other words, when the data analyzer 150 determines that no abnormality is found in the blood pressure data, the data analyzer 150 instructs the data deletion unit 133 to delete the blood pressure data and the data related thereto stored in the data memory 134. More specifically, blood pressure data whose variation in blood pressure is smaller than a threshold, and data related thereto, are to be deleted.

In the above description, biological information is stored and controlled in response to variations in blood pressure. However, biological information may be stored and controlled in response to variations in measurement data of other biological information, such as electrocardiogram and pulse wave.

Next, the data analyzer 150 analyzes how the biological information (here, the blood pressure) and the acceleration data change with time. In the case where the blood pressure data indicates that the blood pressure variation may be a cause of a disease, the data are useful and worthy of attention. The blood pressure variation and acceleration data will be described with reference to FIGS. 5A, 5B, 6A, 6B, 7A, and 7B.

Here, three examples will be described with reference to blood pressure data and acceleration data. The first example is “blood pressure surge due to apnea”, the second example is “respiratory variation”, and the third example is “variation due to a change in body position”. Here, the acceleration sensor is attached to a living body, and accurately expresses changes in the direction of the living body.

In FIGS. 5A, 6A and 7A, curves 501, 601, and 701 represent systolic blood pressures, curves 502, 602, and 702 represent BbB (beat by beat: blood pressures along with a heartbeat), and curves 503, 603, and 703 represent diastolic blood pressures. In FIGS. 5B, 6B, and 7B, curves 505, 605, and 705 represent accelerations in an X direction, curves 506, 606, and 706 represent accelerations in a Y direction, curves 507, 607, and 707 represent accelerations in a Z direction, curves 508, 608, and 708 represent the combined accelerations obtained by combining the accelerations in the X, Y, and Z directions.

### FIRST EXAMPLE

This example is an example of a distinctive blood pressure variation, where the blood pressure variation is due to sleep apnea syndrome (OSAS).

According to the three-axis accelerations shown in FIG. 5B, it is evident that the target living body is almost motionless, for example, even the body position remains unchanged. Referring to FIG. 5A, four apnea periods 504 are regularly shown in this state, and an increase in blood pressure (about 30 mmHg) is measured at all intervals between the apnea periods 504. This is a typical OSAS symptom. The characteristic of OSAS is that the variation of blood pressure at night is large, and at the time of night apnea attack of OSAS, a remarkable blood pressure rise (sleep surge) occurs in tandem with the period from the second half of apnea to the release of apnea. This sleep surge ranges widely from about 20 mmHg to over 100 mmHg, and varies between individuals. Note that the accuracy of the determination as to whether or not this is a sleep time can be improved by acquiring the time from the clocking unit 122 (this also applies to the following two examples).

In the present embodiment, when the cause of the blood pressure variation is determined to be OSAS as described above, the biological information stored in the data memory 134 during the detection period of the blood pressure variation is not targeted for deletion, and the record is retained.

### SECOND EXAMPLE

This example is an example where there is no distinctive variation in blood pressure that is not attributable to an abnormality of the living body; namely, the case where the blood pressure variation is due to a respiratory variation. The respiratory variation is a blood pressure variation synchronized with respiration. This is a phenomenon that occurs even in a resting state, and not a variation in blood pressure caused by a disease.

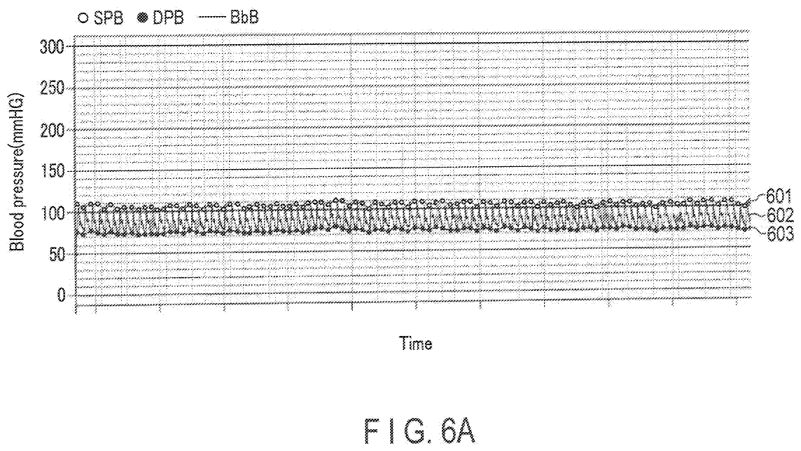

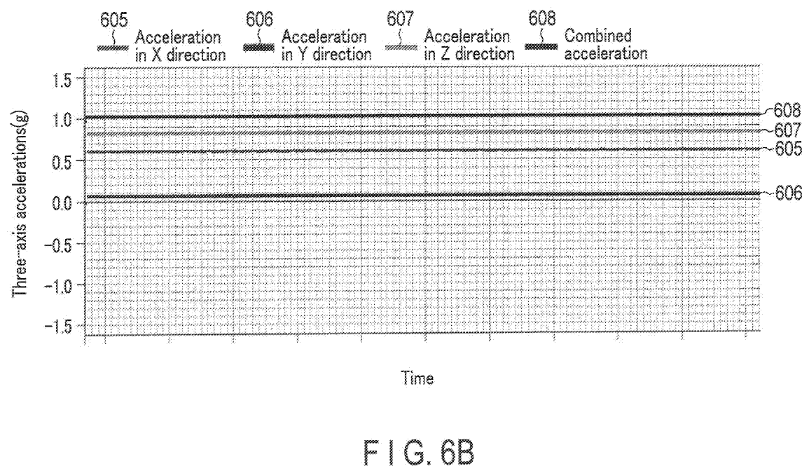

As shown in FIG. 6A, SBP and DBP both show rise and fall in short constant cycles. Considering the variation of BbB in FIG. 6A, it is evident that the systolic blood pressure and the diastolic blood pressure both vary and are completely synchronized for each pulse. Also, the variation of each of systolic blood pressure and diastolic blood pressure is about 5 to 10 mmHg. Furthermore, according to FIG. 6B, the living body is almost motionless. Therefore, the phenomenon of the second example is considered to be a typical respiratory variation.

In the present embodiment, when the blood pressure variation is determined to be the respiratory variation, the biological information stored in the data memory 134 during the blood pressure variation detection period is deleted from the data memory 134 as a deletion target.

### THIRD EXAMPLE

This example is also an example where there is no distinctive variation in blood pressure; namely, the case where the blood pressure variation is due to a change in body position. When the value of an amplitude exceeds a predetermined reference from the values of the amplitudes before and after, the amplitude of the variation of the blood pressure appears as non-periodic, and results from the subject's body position changes during blood pressure measurement.

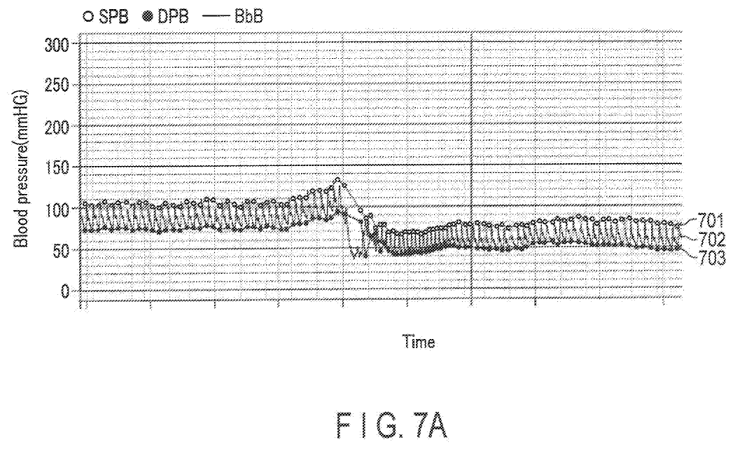

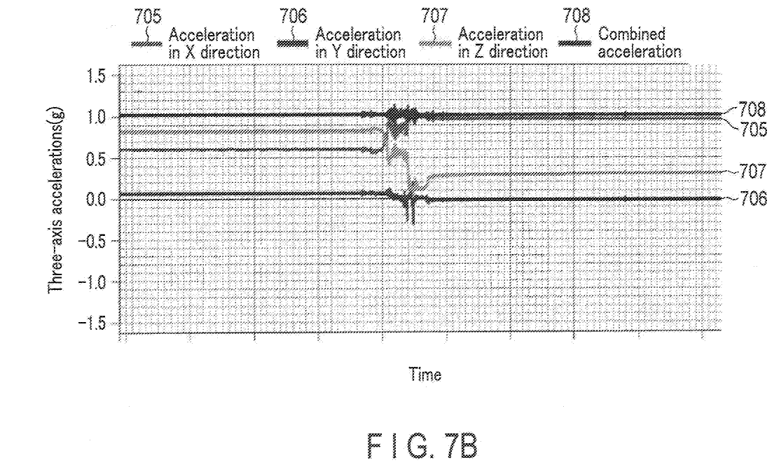

Referring to FIG. 7A, a sharp drop in blood pressure occurs in a central portion of the figure. The three-axis accelerations at the time of the blood pressure drop have rapid changes in the acceleration values in all directions according to FIG. 7B. This change in acceleration is considered to be due to the motion of the living body. Further, since the magnitude of the acceleration in the Z direction and the magnitude of the acceleration in the Y direction are reversed, the change shows that the body direction of the living body is reversed. Therefore, it is considered that the phenomenon of the third example is blood pressure variation due to the typical periodical body position change.

In the present embodiment, when it is determined that the blood pressure variation is a variation due to the change in body position, the biological information stored in the data memory 134 in the detection period of blood pressure variation is deleted from the data memory 134 as a deletion target.

The biological information recording apparatus 100 in the embodiment described above may be modified to include a biological information recording apparatus and a server, and for example, the biological information recording apparatus includes only the data acquisition unit 131, the data memory 134, the data recording unit 132, and the communication unit 160, while the server includes the biological information determination unit 140, the data analyzer 150, the data deletion unit 133, and the communication unit. In this case, the biological information recording apparatus acquires measurement data output from the biological sensor 110 and the acceleration sensor 121 through the data acquisition unit 131, stores the measurement data in the data memory 134, and transmits the data to the server through the communication unit 160. The server determines the blood pressure variation and analyzes its cause by the biological information determination unit 140 and the data analyzer 150 based on the measurement data of the living body and the acceleration transferred from the biological information recording apparatus. Then, if it is determined that the blood pressure variation is a change due to a respiratory variation or body position change, deletion instruction data for deleting the corresponding measurement data is transmitted to the biological information recording apparatus. When the biological information recording apparatus receives the deletion instruction data through the communication unit 160, it deletes from the data memory 134 the measurement data of the period instructed by the data deletion unit 133.

The biological sensor, the acceleration sensor, the clock, and the user input unit 123 need not be provided in the biological information recording apparatus but may be provided in, for example, a wearable terminal.

Also, the biological information recording apparatus may include, for example, the biological sensor 110, the acceleration sensor 121, the clocking unit 122, the user input unit 123, the data acquisition unit 131, a data control unit, and a communication unit, and the server may include a communication unit, a data control unit, the data recording unit 132, the data deletion unit 133, the data memory 134, the data analyzer 150, and the biological information variation determination unit 140.

The data control unit of the biological information recording apparatus transmits the data, obtained by the data acquisition unit 131 from the biological sensor 110, the acceleration sensor 121, and the user input unit 123, to the server via the communication unit.

The communication unit of the server receives the data from the data acquisition unit 131, and the data control unit of the server passes this data to the biological information variation determination unit 140. The biological information variation determination unit 140 determines whether or not the biological information of the living body targeted by the biological information recording apparatus has changed. The data recording unit 132 receives the determination whether or not the biological information of the living body has changed from the biological information variation determination unit 140 via, for example, the data acquisition unit 131. If it is determined that the biological information of the living body has varied, the data recording unit 132 starts recording in the data memory 134 data (biological information such as blood pressure) acquired by the data acquisition unit 131 from the biological sensor 110. After that, if the data analyzer 150 determines that the data is distinctive in temporal variation of biological information (data that is not normal), for example, the data is not deleted but recorded.

The server is, for example, the smart device 200 or the server 300 illustrated in FIG. 2 and may be a separate body independent of the biological information recording apparatus.

With the configuration described above, since the biological information recording apparatus can be made of the minimum configuration, the device to be worn by the user can be compact and lightweight, and easily designed to suit the preferences of the user. Also, since the device part of the biological information recording apparatus is reduced, it can be provided at a lower cost. Furthermore, since the amount of computations to be performed by the biological information recording apparatus is reduced, the amount of memory can be reduced, and the usage rate of the CPU can be reduced.

According to the above embodiment, when the biological information detected by the biological sensor 110 varies outside of a preset normal range, the biological information is stored in the data memory 134. At the same time, if the motion of the living body is detected by the acceleration sensor 121, the above biological information is deleted from the data memory 134. In other words, variations in biological information not caused by the motion of the living body are reliably recorded, whereas variations in biological information caused by the motion of the living body are not recorded. Therefore, it is possible to record the biological information necessary to determine the condition of the living body without omission and not to record unnecessary biological information. As a result, the memory capacity can be reduced.

Also, based on the respiration state of the living body detected by the respiration sensor 113, it is determined whether or not the blood pressure variation is a respiratory variation. If it is determined that the blood pressure variation is a respiratory variation, the relevant biological information stored in the data memory 134 is deleted. Therefore, even if no change in body position is detected, the biological information can be deleted from the data memory 134 if the blood pressure variation is a respiratory variation, which can further reduce the storage amount of the data memory 134.

In the embodiment, if the blood pressure value has varied outside of the normal range, all biological information at this time is once stored in the data memory 134, and it is determined whether or not the stored biological information is caused by an abnormality of the living body. If it is determined that the cause is not an abnormality, the above biological information is deleted from the data memory 134. However, the present invention is not limited to the above embodiment. For example, when it is possible to simultaneously determine whether or not the blood pressure value has varied and whether or not the variation of the blood pressure value is caused by a biological abnormality, the biological information at this time may not be stored. In this way, the step of deleting the biological information once stored can be unnecessary.

The apparatus of the present invention can also be realized by a computer and a program, and the program can be recorded on a recording medium or provided through a network.

Moreover, the above-described apparatuses and their device portions can be implemented either as a hardware configuration or as a combined configuration of hardware resources and software. Used as software of a combined configuration is a program installed in advance from a network or computer-readable recording medium into a computer, and executed by a processor of the computer to cause the computer to realize the functions of the respective devices.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Furthermore, part or all of the above-described embodiments may be described as in the additional descriptions given below; however, the embodiments are not limited thereto.

(Additional Description 1)

A biological information measuring device including a hardware processor and a memory,

the hardware processor being configured to:

acquire biological information detected by a biological sensor,

acquire motion information of the living body detected by a motion sensor;

determine whether or not the biological information has varied outside of a preset normal range; and

record the biological information in the memory if the biological information has varied outside of the normal range and if the motion information of the living body is not detected in a period corresponding to a variation period of the biological information, and not to record the biological information if the biological information has varied outside of the normal range and if the motion information of the living body is detected in the period corresponding to the variation period of the biological information.

(Additional Description 2)

A system including: a biological information recording apparatus that includes a first hardware processor and a first memory; and a server that includes a second hardware processor connected to the biological information recording apparatus via a communication network,

the first hardware processor being configured to:
acquire biological information detected by a biological sensor and record the biological information in the first memory;
transfer the acquired biological information to the server device; and
transfer motion information of the living body detected by a motion sensor to the server device, and

the second hard processor being configured to:
determine whether or not the biological information transferred from the biological information apparatus has varied outside of a preset normal range, and based on the determination result and the motion information transferred from the biological information apparatus, if the motion information of the living body is detected in a period corresponding to a variation period of the biological information, delete the biological information corresponding to the variation period from the memory of the biological information recording apparatus.

(Additional Description 3)

A biological information recording method implemented by a biological information recording apparatus including at least one hardware processor and a memory,

wherein the hardware processor performs control of:
acquiring biological information of a living body from a biological sensor;
acquiring motion information of the living body from a motion sensor;
determining whether or not the biological information has varied outside of a preset normal range;
recording the biological information, if the biological information has varied outside of the normal range and if the motion information of the living body is not detected in a period corresponding to a variation period of the biological information; and
not recording the biological information, if the biological information has varied outside of the normal range and if the motion information of the living body is detected in the period corresponding to the variation period of the biological information. CLAIMS

1. A biological information recording apparatus comprising:
a first acquisition unit configured to acquire biological information detected by a biological sensor;
a second acquisition unit configured to acquire motion information of a living body detected by a motion sensor;
a first determination unit configured to determine whether or not the biological information has varied outside of a preset normal range; and
a recordation control unit configured to record the biological information, if the biological information has varied outside of the normal range and if the motion information of the living body is not detected in a period corresponding to a variation period of the biological information, and fail to record the biological information, if the biological information has varied outside of the normal range and if the motion information of the living body is detected in the period corresponding to the variation period of the biological information,
wherein the biological information is a blood pressure of the living body detected for each pulse along with a heartbeat.

2. The apparatus according to claim 1, wherein the recordation control unit comprises:
a start unit configured to start recording of the biological information if the biological information varies outside of the normal range; and
a deletion unit configured to delete the biological information recorded if the motion information of the living body is detected in the period corresponding to the variation period of the biological information.

3. The apparatus according to claim 1, wherein the biological information is a waveform that changes in synchronization with a cardiac or respiratory cycle of the living body and that is continuously detected.

4. The apparatus according to claim 1, wherein the motion information of the living body is a motion of the living body detected by an acceleration sensor.

5. The apparatus according to claim 1, further comprising a third acquisition unit configured to acquire information representing a respiration state of the living body from a respiration sensor, wherein
the recordation control unit comprises:
a second determination unit configured to determine whether or not the variation of the biological information outside of the normal range is caused by a respiratory variation based on information representing a respiration state acquired from the respiration sensor; and
a first control unit configured to perform a control of not recording the biological information if it is determined that the variation of the biological information outside of the normal range is caused by the respiratory variation.

6. The apparatus according to claim 1, further comprising a third acquisition unit configured to acquire information representing a respiration state of the living body from a respiration sensor, wherein
the recordation control unit comprises:
an apnea period detection unit configured to detect an apnea period of the living body based on the information representing the respiration state acquired from the respiration sensor; and
a second control unit configured to record the blood pressure detected by the blood pressure sensor even if the motion sensor detects that the living body is moving, if it is detected that the blood pressure detected by a blood pressure sensor rises in a respiration period immediately after an end of the apnea period detected by the apnea period detection unit.

7. A biological information recording method comprising:
detecting, by a biological sensor, biological information from a living body;
detecting, by a motion sensor, motion information of the living body;
determining, by a determination unit, whether or not the biological information has varied outside of a preset normal range; and
by a recordation control unit, recording the biological information if the biological information has varied outside of the normal range and if the motion information of the living body is not detected in a period corresponding to a variation period of the biological information, and not recording the biological information if the biological information has varied outside of the normal range and if the motion information of the living body is detected in the period corresponding to the variation period of the biological information,
wherein the biological information is a blood pressure of the living body detected for each pulse along with a heartbeat.

8. A biological information recording apparatus comprising:
a biological sensor configured to detect biological information;
a motion sensor configured to detect motion information of a living body; and
a determination unit configured to determine whether or not the biological information has varied outside of a preset normal range; and
a recordation control unit configured to record the biological information if the biological information has varied outside of the normal range and if the motion information of the living body is not detected in a period corresponding to a variation period of the biological information, and fail to record the biological information if the biological information has varied outside of the normal range and if the motion information of the living body is detected in the period corresponding to the variation period of the biological information,
wherein the biological information is a blood pressure of the living body detected for each pulse along with a heartbeat.

9. A system comprising a biological information recording apparatus, and a server device connected to the biological information recording apparatus via a communication network,
the biological information recording apparatus comprising:
a recording unit configured to acquire and record biological information detected by a biological sensor in a memory;
a first transmission unit configured to transmit the acquired biological information to the server device; and
a second transmission unit configured to transmit motion information of a living body detected by the motion sensor to the server device, and
the server device comprising:
a determination unit configured to determine whether or not the biological information has varied outside of a preset normal range; and
a deletion control unit configured to if the motion information of the living body is detected in a period corresponding to a variation period of the biological information, delete the biological information corresponding to the variation period from the memory of the biological information recording apparatus, based on a determination result by the determination unit and the motion information,
wherein the biological information is a blood pressure of the living body detected for each pulse along with a heartbeat.

10. A non-transitory computer readable medium storing a computer program which is executed by a computer to provide the steps of: acquiring biological information detected by a biological sensor;
acquiring motion information of a living body detected by a motion sensor;
determining whether or not the biological information has varied outside of a preset normal range;
recording the biological information, if the biological information has varied outside of the normal range and if the motion information of the living body is not detected in a period corresponding to a variation period of the biological information; and
failing to record the biological information, if the biological information has varied outside of the normal range and if the motion information of the living body is detected in the period corresponding to the variation period of the biological information,
wherein the biological information is a blood pressure of the living body detected for each pulse along with a heartbeat.
